(19) United States
(12) Patent Application Publication
(10) Pub. No.: US 2013/0065965 A1
(43) Pub. Date: Mar. 14, 2013

(54) DI(4-CHLORO-PHENYLDIGUANIDO) DERIVATIVE WHICH IS FREE OF POTENTIAL GENOTOXICITY AND A PROCESS FOR REDUCING THE RESIDUAL AMOUNT OF P-CHLOROANILINE IN SAID DI(4-CHLORO-PHENYLDIGUANIDO) DERIVATIVE

(71) Applicants: Laura Sanchez Salguero, Girona (ES); Raquel Bou Bosch, Palamós (Girona) (ES); Jordi Bosch I Lladó, Girona (ES)

(72) Inventors: Laura Sanchez Salguero, Girona (ES); Raquel Bou Bosch, Palamós (Girona) (ES); Jordi Bosch I Lladó, Girona (ES)

(73) Assignee: MEDICHEM S.A., SANT JOAN DESPÍ (Barcelona) (ES)

(21) Appl. No.: 13/510,668

(22) PCT Filed: Nov. 18, 2010
(86) PCT No.: PCT/EP10/67782

§ 371 Date: Jun. 26, 2012

Related U.S. Application Data
(60) Provisional application No. 61/262,307, filed on Nov. 18, 2009.
(60) Provisional application No. 61/373,449, filed on Aug. 13, 2010.

(51) Int. Cl. A61K 31/155; C07C 279/18

(52) U.S. Cl. 514/635; 564/235

(57) ABSTRACT

The invention relates to a process for reducing the residual amount of p-chloroaniline in chlorhexidine. Also, the invention relates to a process for preparing chlorhexidine, or a pharmaceutically acceptable salt thereof, which is free of potential genotoxicity. In addition, the invention refers to the said chlorhexidine, or a pharmaceutically acceptable salt thereof, which is free of potential genotoxicity. Further, the invention relates to an analytical HPLC method for the determination of potentially genotoxic impurities in samples of chlorhexidine, or of a pharmaceutically acceptable salt thereof. The invention also relates to stabilized chlorhexidine digluconate salt free of potential genotoxicity in aqueous solution, and to a method for stabilizing chlorhexidine digluconate salt free of potential genotoxicity in aqueous solution.

This application claims the benefit of the U.S. Provisional Patent Application Ser. No. 61/262,307 filed on 18 Nov. 2009 and U.S. Provisional Patent Application Ser. No. 61/373,449 filed on 13 Aug. 2010.

### BACKGROUND ART

Chlorhexidine (compound I) is the international common accepted name for 1-[amino-[6-[amino-[amino-(4-chlorophenyl)aminomethylidene]aminomethylidene]amino-hexylimino]methyl]imino-N-(4-chlorophenyl)-methanediamine [also known as 1,6-di(4′-chloro-phenyldiguanido)hexane], and has an empirical formula of C₂₂H₃₀Cl₂N₁₀ and a molecular weight of 505.45.

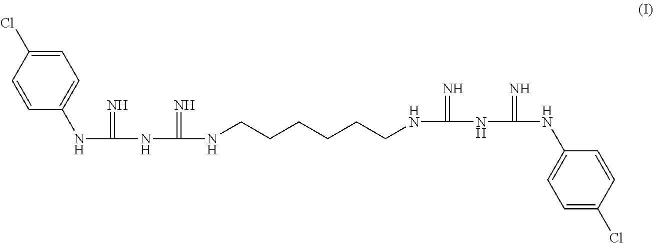 [SMILES: N=C(NCCCCCCNC(=N)NC(=N)Nc1ccc(Cl)cc1)NC(=N)Nc1ccc(Cl)cc1]

Chlorhexidine is a well-known chemical antiseptic and disinfectant which, due to its poor solubility, it is mainly used in one of its known salt forms (i.e. digluconate, diacetate or dihydrochloride). Chlorhexidine salts are antibacterial agents, used for human and animal disinfection. Also, chlorhexidine salts have a very wide range of antimicrobial activity, being effective either against gram-positive or gram-negative organisms. In addition, chlorhexidine salts have fungicidal and sporicidal effect. Thus, chlorhexidine products are used for a number of applications such as dairy hygiene applications, oral antiseptic applications, hand and skin disinfection, general disinfection (equipment, surfaces and textiles), etc.

U.S. Pat. No. 2,684,924 discloses the preparation of chlorhexidine in its dihydrochloride salt. More precisely, in Example 1 of this reference the dihydrochloride salt of chlorhexidine is prepared by reacting hexamethylene bis-dicyandiamide (compound II) with p-chloroaniline hydrochloride (compound III) in the presence of β-ethoxyethanol (See Scheme 1).

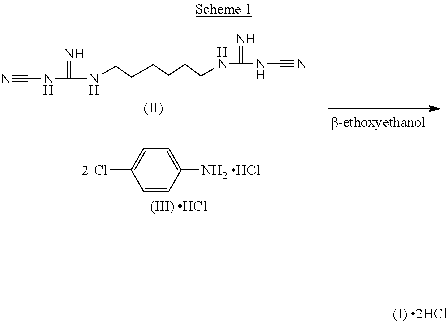

Since p-chloroaniline (compound III) is used as an intermediate for the synthesis of chlorhexidine base and its salts, it is likely to be present as an impurity not only in the chlorhexidine base and salts as such, but also in the finished product. Further, it is known that chlorhexidine salts are likely to decompose to also produce trace amounts of compound (III). In this regard, in the U.S. Pharmacopoeia (First Supplement to the USP 33-NF 28 Reissue, USP Monographs: Chlorhexidine Gluconate Solution) it is described that the digluconate salt of chlorhexidine in aqueous solution should be preserved in tight containers, protected from light, and at controlled room temperature (i.e. generally understood as 25° C.).

F. L. Rose et al. in J. Chem. Soc., 1956, 4422, describe that chlorhexidine base (I) can be simply obtained by adding a hot aqueous solution of sodium hydroxide to the dihydrochloride salt of chlorhexidine, (I).2HCl. The chlorhexidine base obtained therein needs to be purified by recrystallization from methanol, to obtain a chlorhexidine in form of colourless needles and showing a melting point of 133.5-134° C. In the applicants' hands, the recrystallization process described in this reference is not efficient and suitable for industrial scale, since it requires the use of large volumes of methanol per gram of chlorhexidine (i.e. 30 mL of methanol/g of chlorhexidine) and provides the product with moderate yield (i.e. about 61%).

JP Patent No. 04164061A discloses that the chlorhexidine base obtained by F. L. Rose et al. in J. Chem. Soc., 1956, 4422, shows a low purity profile (i.e. % purity of about 62.8, and melting point of about 129-131° C.), and remarks that the recrystallization process described therein is unsatisfying as an industrial process, and describes a new method for preparing the same. More precisely, the chlorhexidine base described in this reference is prepared by (i) treating chlorhexidine dihydrochloride (prepared from compounds (III) and (II) as described in U.S. Pat. No. 2,684,924) with sodium hydroxide in the presence of a solvent comprising a lower alcohol and water, preferably comprising a 40-95% concentration of the lower alcohol, (ii) precipitating and filtering the chlorhexidine base, and (iii) washing the chlorhexidine base precipitate with an alcoholic aqueous solution. The chlorhexidine base obtained in this reference has a purity ranging from 96-99.2%, and a melting point of about 132.2-134° C.

BR Patent Application No. PI 9300129A describes a process for preparing chlorhexidine dihydrochloride by reacting compound (II) with compound (III) and the obtained salt shows a residual concentration (or amount) of p-chloroaniline of about 2000 ppm. The chlorhexidine base is in turn prepared by (i) treatment of the chorhexidine dihydrochloride with a sodium hydroxide solution, in the presence of a mixture of water and isopropanol as a solvent, (ii) centrifugation of the final solution, and (iii) washing the chlorhexidine base precipitate with water and methanol. The obtained chlorhexidine base shows a residual concentration (or amount) of p-chloroaniline of about 1000 ppm. Finally, the respective digluconate and diacetate salts are prepared by controlled reaction of the chlorhexidine base with gluco-delta-lactone and glacial acetic acid, respectively. The obtained digluconate salt shows a residual concentration (or amount) of p-chloroaniline of about 500 ppm.

Since chlorhexidine base shows a very low solubility profile, the procedures described in the prior art make use of washing procedures in order to purify said compound. It is understood that when washing the chlorhexidine with a solvent, the purification process works similar to a digestion-based purification. That is, by means of a short time of contact of the chlorhexidine with the solvent, the impurity is dissolved and removed by the solvent whereas the product remains as a solid. Further, when the washing procedures described in the prior art are carried out at industrial scale, the said washing procedures can be extended to digestion-based purifications since the time of contact of the chlorhexidine with the solvent becomes significant. The applicants have found that the said washing- or digestion-based purifications of chlorhexidine described in the prior art are low effective in order to reduce the characteristic residual content of (III), since they show a low percentage of reduction (i.e. less than about 52% of reduction per digestion step). This feature increases the cost of these purifications, especially at industrial scale.

Also, it is known that compound (III) is a toxic substance which shows potential genotoxic properties which is an indication that the material may have mutagenic and carcinogenic potential, and hence to date its presence in chlorhexidine salts, and hence in chlorhexidine base, is currently restricted by the European Pharmacopoeia to a limit of 500 ppm (i.e. 0.05%). Therefore, to date the presence of compound (III) in chlorhexidine salts, and hence in chlorhexidine base, for use in pharmaceuticals needs to be simply controlled in order to fulfill the general acceptance of not more than 0.05%.

### SUMMARY OF THE INVENTION

The invention relates to a process for reducing the residual amount of p-chloroaniline in chlorhexidine. Also, the invention relates to a process for preparing chlorhexidine, or a pharmaceutically acceptable salt thereof, which is free of potential genotoxicity. In addition, the invention refers to the said chlorhexidine, or a pharmaceutically acceptable salt thereof, which is free of potential genotoxicity. Further, the invention relates to an analytical HPLC method for the determination of potentially genotoxic impurities in samples of chlorhexidine, or of a pharmaceutically acceptable salt thereof. The invention also relates to stabilized chlorhexidine digluconate salt free of potential genotoxicity in aqueous solution, and to a method for stabilizing chlorhexidine digluconate salt free of potential genotoxicity in aqueous solution.

### BRIEF DESCRIPTION OF THE DRAWINGS

FIG. 1 shows the degradation rate towards p-chloroaniline and two individual values, respectively, of two different chlorhexidines free of potential genotoxicity (i.e. chlorhexidine having less than 0.15% of impurities B-G, and having a total impurities content less than 1.5%; and chlorhexidine having more than 0.15% of impurities B-G, and having a total impurities content between 1.5-3.0%, respectively) stored in a methanol:acetic acid 8:1 (v/v) solution at 40° C.

### DETAILED DESCRIPTION OF THE INVENTION

In a first aspect, the present invention provides a process for reducing the residual amount of p-chloroaniline, (compound of formula III),

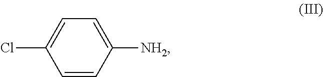

in chlorhexidine, (compound of formula I),

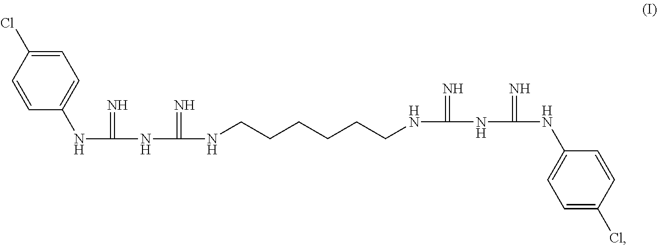 [SMILES: N=C(NCCCCCCNC(=N)NC(=N)Nc1ccc(Cl)cc1)NC(=N)Nc1ccc(Cl)cc1]

said process comprising: (i) suspending the chlorhexidine in at least one organic solvent essentially free of water, and (ii) isolating the chlorhexidine from the suspension.

The washing- or digestion-based purifications of chlorhexidine described in the prior art are low effective in order to reduce the characteristic residual content of (III). The applicants have surprisingly found that the key feature which makes these prior art purifications low effective is the presence of water in the purification process. Precisely, when conducting digestion purifications of chlorhexidine by following the teachings of the washing- or digestion-based processes used in the prior art (which all contain water as a purification co-solvent), the obtained chlorhexidine base has a moderate percentage of reduction of (III) (i.e. less than about 52% per digestion step. See comparative Examples 2 and 3). In fact, this percentage of reduction should be even lower if the purification should be limited to a washing step, since the time of contact with the solvent will be lower. Further, when the digestion purification process was carried out in only water, the percentage of reduction of (III) was almost inefficient (i.e. of about 6% per digestion step. See comparative Example 1). In addition, when the water-based digestion comprises refluxing the suspension mixture, the chlorhexidine surprisingly decomposes and the content of residual (III) dramatically increases instead of decreasing (i.e. about 16% of increasing. See comparative Example 6). Thus, the presence of water in the digestion purification, is not only low effective, but is also undesirable since it has been shown to increase the presence of the residual (III) in chlorhexidine. Thus, the inventors have found that when the chlorhexidine base is purified by means of the process of the invention above (i.e. digestion in at least one organic solvent essentially free of water), the percentage of reduction of the residual content of (III) is highly effective (i.e. between 60-89% per digestion step).

The at least one organic solvent of step (i) of the process of the invention above is preferably selected from the group consisting of (C₃-C₆)-ketone solvents, C₁-C₅ alcohol solvents and mixtures thereof. Examples of such solvents include: methanol, ethanol, isopropanol, n-propanol, n-butanol, t-butanol, isoamyl alcohol, acetone, methylethylketone, methylisobutylketone, among others. More preferably the at least organic solvent is acetone, methanol, isopropanol, or mixtures thereof, and even more preferably the at least one organic solvent is methanol or an acetone/methanol mixture, since the percentage of reduction of the residual content of (III) in these latest solvents can be higher than 80% per digestion step. Also, the acetone/methanol mixture preferably contains a (v/v) percentage of acetone of between 5-30%.

It should be noted that the term “essentially free of water” as described in step (i) of the process of the invention above means that the at least one organic solvent comprises less than 1% concentration of water, preferably less than 0.5% concentration of water, more preferably less than 0.1% concentration of water, and even more preferably the at least one organic solvent does not contain water.

The volume of solvent in respect of chlorhexidine is preferably less than 20 mL/g, more preferably less than 10 mL/g, and even more preferably less than 5 mL/g.

The suspending the chlorhexidine in at least one organic solvent essentially free of water of step (i) of the process above can be carried out either at room temperature or at a higher temperature, e.g. at reflux temperature. Preferably, the suspending the chlorhexidine in at least one organic solvent essentially free of water of step (i) of the process above is carried out at reflux temperature, since in this case increases the percentage of reduction of the residual content of (III). Then, when the process of step (i) is carried out at a temperature higher than room temperature, the process can optionally comprise an additional step of cooling the hot suspension.

The suspending the chlorhexidine in at least one organic solvent essentially free of water of step (i) of the process above can be carried out for different suitable periods of time, not being necessarily restricted to either large or short periods of time.

The isolating the chlorhexidine from the suspension of step (ii) of the process above can be carried out by different methods known in the art, such as filtering the suspension, decanting the solvent from the suspension, or spray-drying the suspension. Preferably, the isolating step is carried out by filtering the suspension, since it is a simple procedure which is suitable for industrial scale.

The process of the invention above may be repeated as many times as desired in order to obtain a desired reduction of the residual content of p-chloroaniline (III). Namely, the chlorhexidine isolated in step (ii) of the process of the invention above typically provides chlorhexidine having a residual concentration of p-chloroaniline of less than about 500 ppm, preferably less than about 300 ppm, more preferably less than about 100 ppm, even more preferably less than about 50 ppm, and still even more preferably equal to or less than about 43 ppm, by HPLC. Also, the chlorhexidine obtained by this process typically shows a total impurities content of less than 0.1%, and preferably less than 0.05%, by HPLC. Further, the chlorhexidine obtained by this process typically shows a melting point value higher than the melting point values described in the prior art for chlorhexidine base (i.e. 134.6-135.4° C.), which clearly demonstrates that the said product shows a higher purity profile.

The process of the invention above can be carried out not only for chlorhexidine in its non-protonated form (i.e. chlorhexidine base), but also for chlorhexidine in any of its known salts. If the reduction of the content of compound (III) is to be carried out for a salt of chlorhexidine, the process of the invention above then comprises an initial step of isolating chlorhexidine in its free base form from the salt form. The isolating can be carried out by any method known in the art for isolating chlorhexidine base from a salt of chlorhexidine.

In addition, although it is known that compound (III) is a toxic substance which shows potential genotoxic properties and its presence in chlorhexidine salts, and hence in chlorhexidine base, is currently restricted to a limit of 500 ppm (i.e. 0.05%), the present inventors have also found that the acceptable limit for compound (III) in chlorhexidine, or its pharmaceutically acceptable salts, needs to be significantly narrowed to a value much lower than the current 0.05% general acceptance.

Namely, the applicants have also found that the presence of compound (III) in chlorhexidine, and its pharmaceutically acceptable salts, needs to be tightly controlled and restricted in order to fulfill the permitted daily dose for potential genotoxic impurities present in a pharmaceutical product. In this regard, a dose of 1.5 micrograms/day has been described as the acceptable level for genotoxic impurities in pharmaceuticals. Thus, the concentration of a potential genotoxic impurity considered acceptable in a pharmaceutical product can be calculated according to the following formula: maximum concentration of the potential genotoxic impurity (ppm)=1.5 μg/active ingredient daily dose (in g). Since there are a number of chlorhexidine salt pharmaceutical products, the maximum recommended dose may vary for each product. After considering the formulations containing chlorhexidine in any of its salts wherein the maximum daily dose has the highest value [i.e. the oral formulations containing chlorhexidine dihydrochloride for the treatment of mouth and throat disorders, which typically have a maximum recommended daily dose of 40 mg of chlorhexidine dihydrochloride salt], the present inventors have calculated that the highest possible maximum dose for chlorhexidine in its free base form is of about 35 mg daily. Thus, the present inventors have found that chlorhexidine and it salts, for use in pharmaceuticals, which hence may be free of potential genotoxicity, will require a maximum concentration of compound (III) of not more than about 40-50 ppm, preferably of not more than about 41-49 ppm, preferably of not more than about 42-48 ppm, preferably of not more than about 42-44 ppm, and preferably of not more than about 43 ppm. Also, the applicants provide a process for preparing said chlorhexidine and its salts. The term “ppm” as used in this application means parts of compound (III) per million parts chlorhexidine base. In other words, it relates to the concentration of compound (III) in μg per g of chlorhexidine base. The respective concentration of compound (III) relating to chlorhexidine salts can be calculated accordingly.

Thus, in another aspect, the invention provides a process for preparing chlorhexidine, or a pharmaceutically acceptable salt thereof, which is free of potential genotoxicity, said process comprising: (i) providing chlorhexidine; (ii) measuring the concentration of p-chloroaniline in the chlorhexidine, (iii) if the concentration of p-chloroaniline in the chlorhexidine is higher than about 40-50 ppm, preferably higher than about 41-49 ppm, preferably higher than about 42-48 ppm, preferably higher than about 42-44 ppm, and preferably higher than about 43 ppm, by HPLC, carrying out, at least once, the process of the invention above for reducing the amount of p-chloroaniline in chlorhexidine; and (iv) optionally, preparing a pharmaceutically acceptable salt of chlorhexidine.

The providing chlorhexidine of step (i) of the process above for preparing chlorhexidine, or a pharmaceutically acceptable salt thereof, which is free of potential genotoxicity, preferably comprises either (i)(a) synthesizing chlorhexidine directly in its free base form; or (i)(b) isolating chlorhexidine in its free base form from a pharmaceutically acceptable salt of chlorhexidine. The synthesizing can be carried out by any method known in the art. The isolating can be carried out by any method known in the art for isolating chlorhexidine base from a pharmaceutically acceptable salt of chlorhexidine.

The pharmaceutically acceptable salt of chlorhexidine of step (iv) of the process above for preparing chlorhexidine, or a pharmaceutically acceptable salt thereof, which is free of potential genotoxicity, is preferably the mono- or diacetate salt, the mono- or dihydrochloride salt, or the mono- or digluconate salt of chlorhexidine.

The process above for preparing chlorhexidine, or a pharmaceutically acceptable salt thereof, which is free of potential genotoxicity, may optionally comprise an additional step of (v) admixing the chlorhexidine, or a pharmaceutically acceptable salt thereof, with at least one pharmaceutically acceptable carrier and/or with at least one additional active pharmaceutical ingredient. The said at least one pharmaceutically acceptable carrier can be any pharmaceutically acceptable carrier known in the art suitable for preparing a pharmaceutical formulation of chlorhexidine, or salts thereof. Preferably, the pharmaceutically acceptable carrier is benzalkonium chloride, menthol, ethanol, water, or mixtures thereof. The said at least one additional active pharmaceutical ingredient can be any additional active pharmaceutical ingredient known in the art suitable for preparing a pharmaceutical formulation of chlorhexidine, or salts thereof. Preferably, the at least one additional active pharmaceutical ingredient is benzocaine, tirotricine, lidocaine, enoxolone, or mixtures thereof.

In another further aspect, the present invention refers to chlorhexidine, or a pharmaceutically acceptable salt thereof, which is free of potential genotoxicity, and wherein the said chlorhexidine, or a pharmaceutically acceptable salt thereof, has a concentration of equal to or less than about 40-50 ppm, preferably of equal to or less than about 41-49 ppm, preferably of equal to or less than about 42-48 ppm, preferably of equal to or less than about 42-44 ppm, and preferably of equal to or less than about 43 ppm of p-chloroaniline, by HPLC. Also, the said chlorhexidine, or a pharmaceutically acceptable salt thereof, which is free of potential genotoxicity preferably shows a total impurities content of less than 0.1%, and preferably less than 0.05%, of percentage area by HPLC. Further, the said chlorhexidine, or a pharmaceutically acceptable salt thereof, which is free of potential genotoxicity shows a melting point value higher than the melting point values described in the prior art for chlorhexidine base (i.e. 134.6-135.4° C.), which clearly demonstrates that the said product shows a higher purity profile.

In another aspect, the present inventors have also found that in order to obtain a chlorhexidine, or a pharmaceutically acceptable salt thereof, which is free of potential genotoxicity, not only the presence of compound (III) as such needs to be controlled, but also the percentage of certain impurities present in the chlorhexidine, or pharmaceutically acceptable salt thereof, is preferably significantly narrowed to a value much lower than the current general acceptance for impurities in chlorhexidine, or pharmaceutically acceptable salts thereof, which is currently broadly limited to a total value of either 2.5% or 3.0%, for the different chlorhexidine salts described in the European Pharmacopeia.

Namely, the inventors have identified a number of impurities other than p-chloroaniline (compound III) which may be also present in chlorhexidine, and in its pharmaceutically acceptable salts, the structure of which has been confirmed after isolation and fully characterization (See Table 1 below. Impurities B-G). The above detected impurities which may be present in chlorhexidine, and in its pharmaceutically acceptable salts, contain at least one 4-chlorophenylamino moiety in their structure and consequently, in the same manner as chlorhexidine, are likely to degrade into the potentially genotoxic compound (III). The said chlorhexidine impurities containing at least one 4-chlorophenylamino moiety in their structure can be defined by the compound of Markush formula IV,

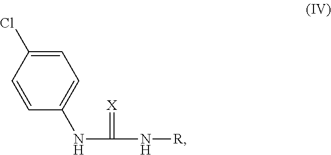

wherein X is NH or O, and R is H, a carboxamidino group, or a carboxamido group.

In this regard, the inventors have found that samples of chlorhexidine, and of pharmaceutically acceptable salts thereof, having a higher presence of said impurities of formula IV, or salts thereof, show worse properties as compared with samples of chlorhexidine, or pharmaceutically acceptable salts thereof, with lower content of said impurities, and principally show a higher tendency to produce compound (III) as a degradation by-product. Thus, the applicants have also found that the presence of said impurities of formula IV, in chlorhexidine, or its pharmaceutically acceptable salts, is preferably needed to be tightly controlled and restricted in order to obtain the chlorhexidine, or pharmaceutically acceptable salt thereof, of the invention which is free of potential genotoxicity and which has a maximum concentration of compound (III) of equal to or less than about 40-50 ppm, preferably of equal to or less than about 41-49 ppm, preferably of equal to or less than about 42-48 ppm, preferably of equal to or less than about 42-44 ppm, and preferably of equal to or less than about 43 ppm.

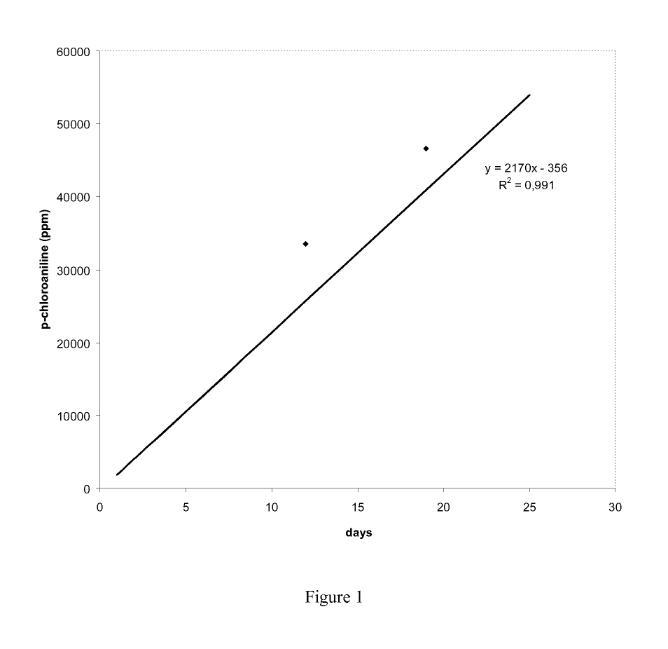

Since the inventors have found that impurities of formula (IV), or salts thereof, are chemically less stable than chlorhexidine, or salts thereof, and that for that reason they are more likely to degrade into the potentially genotoxic compound (III), and in the absence of toxicological studies describing the acceptable risk level of said impurities in chlorhexidine, the present inventors propose that their presence as impurities in chlorhexidine, or in its pharmaceutically acceptable salts, should be preferably restricted, at least, according to the general acceptance limit given by regulatory bodies for impurities in active pharmaceutical ingredients (i.e. 0.15%). Namely, the presence of impurities of formula (IV) in chlorhexidine, or salts thereof, should be preferably reduced to a maximum concentration of not more than 0.15% for each impurity of formula (IV) [e.g. B-G]. In this regard, the inventors have carried out a p-chloroaniline degradation study for samples of chlorhexidine base in solution (i.e. methanol:acetic acid 8:1 (v/v)), which are free of potential genotoxicity as herein above described (i.e. having less than 40-50 ppm of (III)), and have observed that samples of chlorhexidine free of potential genotoxicity as herein above described showing a content of compounds B-G [i.e. impurities of formula (IV)] higher than 0.15%, and showing a total impurities content between 1.5-3.0%, show a higher tendency to degrade to genotoxic p-chloroaniline (compound (III)), after at least 12 days of storage under 40° C., as compared with samples of chlorhexidine free of potential genotoxicity as herein above described showing not more than 0.15% of compounds B-G, and a total impurities content less than 1.5% (See Example 11 and FIG. 1). Therefore, the applicants have also found that chlorhexidine, and its salts, for use in pharmaceuticals, which hence may be free of potential genotoxicity, will not only require a maximum concentration of compound (III) of not more than about 40-50 ppm (i.e. not more than about 0.005%), but also will preferably require a maximum concentration of each impurity of formula (IV) [e.g. B-G] of not more than 0.15%. Preferably, the presence of each of the impurities of formula (IV) in chlorhexidine should be restricted to the maximum concentration found in the present invention for compound (III) in chlorhexidine (i.e. 0.005%). Also, taking into account the calculated maximum concentration for each of the potentially genotoxic impurities that are likely to be present in chlorhexidine, the present inventors also propose that the total impurities limit for chlorhexidine, or pharmaceutically acceptable salts thereof, which may be free of potential genotoxicity should be also preferably restricted to a maximum of 1.5%, instead of the currently accepted 2.5% or 3.0%.

TABLE 1

| Impurity | Structure |
|---|---|
| A (i.e., compound III) | 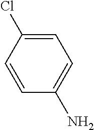 |
| B | 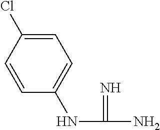 [SMILES: N=C(N)Nc1ccc(Cl)cc1] |
| C | 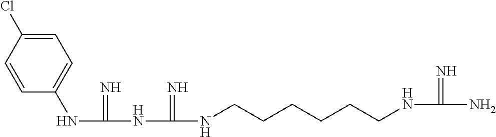 [SMILES: N=C(N)NCCCCCCNC(=N)NC(=N)Nc1ccc(Cl)cc1] |
| D | 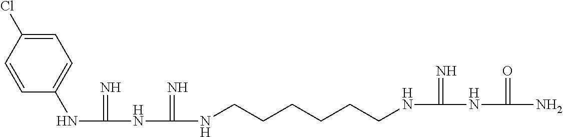 [SMILES: N=C(NCCCCCCNC(=N)NC(N)=O)NC(=N)Nc1ccc(Cl)cc1] |
| E | 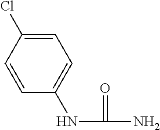 [SMILES: NC(=O)Nc1ccc(Cl)cc1] |
| F | 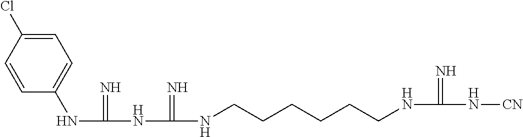 [SMILES: N#CNC(=N)NCCCCCCNC(=N)NC(=N)Nc1ccc(Cl)cc1] |
| G | 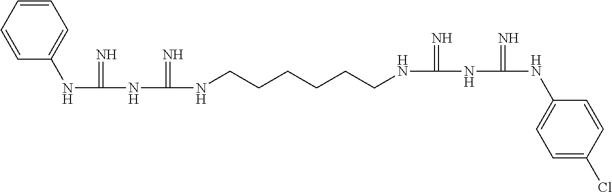 [SMILES: N=C(NCCCCCCNC(=N)NC(=N)Nc1ccc(Cl)cc1)NC(=N)Nc1ccccc1] |

Thus, in another aspect, the present invention provides chlorhexidine, or a pharmaceutically acceptable salt thereof, which is free of potential genotoxicity, and wherein the said chlorhexidine, or a pharmaceutically acceptable salt thereof, preferably has a content of equal to or less than 0.15%, preferably equal to or less than 0.10%, more preferably equal to or less than 0.05%, and even more preferably equal to or less than 0.005%, of percentage area by HPLC of each of the compounds of formula (IV) [e.g. B-G].

The process of the invention above for reducing the residual amount of p-chloroaniline (compound III) in chlorhexidine is also suitable for keeping the total impurities content of chlorhexidine below the limits of the invention described above, and specifically is suitable for keeping the content of each of the compounds of formula (IV) [e.g. B-G] in chlorhexidine below the limits of the invention described above.

In a preferred further aspect, the chlorhexidine, or a pharmaceutically acceptable salt thereof, which is free of potential genotoxicity of the invention, is characterized by having (a) a concentration of equal to or less than about 40-50 ppm, preferably of equal to or less than about 41-49 ppm, preferably of equal to or less than about 42-48 ppm, preferably of equal to or less than about 42-44 ppm, and preferably of equal to or less than about 43 ppm of p-chloroaniline, by HPLC, and (b) a content of equal to or less than 0.15%, preferably equal to or less than 0.10%, more preferably equal to or less than 0.05%, and even more preferably equal to or less than 0.005%, of percentage area by HPLC of each of the compounds of formula (IV) [e.g. B-G].

Thus, in yet another aspect, the steps (ii) and (iii) of the process of the invention above for preparing chlorhexidine, or a pharmaceutically acceptable salt thereof, which is free of potential genotoxicity, comprise: (ii) (a) measuring the concentration of p-chloroaniline in the chlorhexidine; preferably (b) measuring the content of one or more compounds of formula IV [e.g. B-G], and preferably (c) measuring the total impurities content, and (iii) if the concentration of p-chloroaniline in the chlorhexidine is higher than about 40-50 ppm, preferably higher than about 41-49 ppm, preferably higher than about 42-48 ppm, preferably higher than about 42-44 ppm, and preferably higher than about 43 ppm, by HPLC, and, preferably, if the content of any of compounds of formula (IV) [e.g. B-G] is higher than 0.15%, preferably higher than 0.10%, more preferably higher than 0.05%, and even more preferably higher than 0.005%, of percentage area by HPLC, and, preferably, if the total impurities content is higher than 1.5%, by HPLC, carrying out, at least once, the process of the invention above for reducing the amount of p-chloroaniline and of compounds of formula (IV) and of total impurities in chlorhexidine.

The pharmaceutically acceptable salt of chlorhexidine which is free of potential genotoxicity of the invention is preferably the mono- or diacetate salt, the mono- or dihydrochloride salt, or the mono- or digluconate salt of chlorhexidine.

The chlorhexidine, or a pharmaceutically acceptable salt thereof, free of potential genotoxicity of the invention can be admixed with at least one pharmaceutically acceptable carrier and/or with at least one additional active pharmaceutical ingredient. Thereby, a pharmaceutical formulation comprising chlorhexidine, or a pharmaceutically acceptable salt thereof, according to the present invention is provided. The said at least one pharmaceutically acceptable carrier can be any pharmaceutically acceptable carrier known in the art suitable for preparing a pharmaceutical formulation of chlorhexidine, or salts thereof. Preferably, the pharmaceutically acceptable carrier is benzalkonium chloride, menthol, ethanol, water, or mixtures thereof. The said at least one additional active pharmaceutical ingredient can be any additional active pharmaceutical ingredient known in the art suitable for preparing a pharmaceutical formulation of chlorhexidine, or salts thereof. Preferably, the at least one additional active pharmaceutical ingredient is benzocaine, tirotricine, lidocaine, enoxolone, or mixtures thereof.

The inventors have additionally surprisingly found that the stability of the chlohexidine digluconate of the present invention in aqueous solution, which is free of potential genotoxicity, can be dramatically affected by the temperature of storage. In the U.S. Pharmacopoeia (First Supplement to the USP 33-NF 28 Reissue, USP Monographs: Chlorhexidine Gluconate Solution) it is described that the digluconate salt of chlorhexidine in aqueous solution should be preserved in tight containers, protected from light, and at controlled room temperature (i.e. about 25° C.). In this regard, the present inventors have observed that a 20% (w/v) aqueous solution of the chlorhexidine digluconate of the invention which is free of potential genotoxicity (i.e. having a content of p-chloroaniline lower than 40-50 ppm, with respect to the chlorhexidine base) can become moderately potentially genotoxic (i.e. showing a content of p-chloroaniline dramatically close to higher than 40-50 ppm) after 3 months of storage at 25° C., and highly potentially genotoxic (i.e. showing a content of p-chloroaniline much higher than 40-50 ppm) after 3 months of storage at 40° C. Further, the percentage of increasing of the degradation by-product p-chloroaniline when storing the aqueous solution at 40° C. is of 4233%, and of 1533% when stored at 25° C., for at least 3 months. Conversely, when storing the aqueous solution of chlorhexidine digluconate of the invention at 15° C., the free-genotoxic stability (i.e. a content of p-chloroaniline lower than 40-50 ppm) is kept at low values for at least 3 months (See Example 12). Further, the percentage of increasing of the degradation by-product p-chloroaniline under these later storage conditions is only of 433%, a value which is unexpectedly dramatically lower than the percentage of increasing of the degradation by-product p-chloroaniline obtained when storing the same sample at 40° C. or at 25° C.

Accordingly, the present invention provides an aqueous solution of the digluconate salt of chlorhexidine, preferably chlorhexidine which is free of potential genotoxicity as described hereinbefore, which is stabilized by storage at a temperature below 25° C., preferably below 23° C., preferably below 21° C., preferably below 19° C., preferably below 17° C., and preferably below 15° C., for at least 3 months.

The term stabilized digluconate salt of chlorhexidine in aqueous solution as used herein is meant to refer to digluconate salt of chlorhexidine showing a percentage of increasing of degradation by-product p-chloroaniline of less than 1500%, preferably less than 1000%, and preferably less than 500%, when stored in an aqueous solution showing a (w/v) percentage concentration of chlorhexidine digluconate of 90-5%, preferably 80-10%, preferably 50-15%, preferably 20%, and at a temperature below 25° C. as above described, for at least 3 months. The term stabilized digluconate salt of chlorhexidine which is free of potential genotoxicity as used herein is meant to refer to digluconate salt of chlorhexidine which shows a content of p-chloroaniline, with respect to the chlorhexidine base, lower than 40-50 ppm, preferably lower than 43 ppm as herein above described, when stored in an aqueous solution as above described and at a temperature below 25° C. as above described, for at least 3 months.

The stabilized aqueous solution of the digluconate salt of chlorhexidine as described above is preferably stored in the absence of light. Also, the said stabilized aqueous solution is preferably stored in an enclosed plastic or metal container or packaging.

In another aspect, the invention relates to a method for storing or packaging an aqueous solution of the digluconate salt of chlorhexidine, preferably chlorhexidine which is free of potential genotoxicity as described herein before, wherein the storing or packaging procedure is carried out at a temperature below 25° C., preferably below 23° C., preferably below 21° C., preferably below 19° C., preferably below 17° C., and preferably below 15° C.

In yet another aspect, the invention relates to a method for stabilization of an aqueous solution of the digluconate salt of chlorhexidine, preferably chlorhexidine which is free of potential genotoxicity as described herein before, said method comprising storing or packaging the aqueous solution at a temperature below 25° C., preferably below 23° C., preferably below 21° C., preferably below 19° C., preferably below 17° C., and preferably below 15° C.

Further, the control of the presence of compound (III) in chlorhexidine at the new low limit found by the present inventors represents a significant challenge from an analytical point of view. In this regard, the present inventors provide an HPLC method (HPLC method 1) which is suitable for determining the content of (III) in chlorhexidine at such low limits. Also, the HPLC method 1 provided in the present invention is useful not only for determining the content of (III) in chlorhexidine, but also for determining the total impurities content of said chlorhexidine.

In yet another aspect, the present invention provides an analytic HPLC method 1 for the determination of potentially genotoxic impurities in samples of chlorhexidine, or a pharmaceutically acceptable salt thereof, characterized in that said analytic method comprises a High Performance Liquid Chromatography (HPLC) apparatus which can detect at least a concentration of p-chloroaniline equal to or less than about 40-50 ppm, preferably equal to or less than about 43 ppm, the method comprising using a C₁₈ column having equal to or less than 5 μm of particle size and a mobile phase comprising a mixture of 1-octanesulfonic acid sodium salt solution/glacial acetic acid/methanol, wherein the mixture comprises less than 60% of methanol. The method is generally carried out at a temperature higher than 25° C., preferably, equally or higher than 30° C.

In another aspect, the control of the content of compounds of formula (IV) [e.g. B-G] in chlorhexidine at the limits of the present invention can be carried out using the HPLC method 2 provided in the present invention, characterized in that said analytic method comprises a C₁₈ column having equal to or less than 10 μm of particle size and a mobile phase comprising a mixture of 1-octanesulfonic acid sodium salt solution in water/glacial acetic acid/methanol.

Within the scope of this application, it should be noted that when chlorhexidine, or chlorhexidine base, is mentioned, any pharmaceutically acceptable salt of chlorhexidine is also considered.

Also, it should be noted that the term “about” as used herein for ppm values is meant to convey ±5 ppm.

### SPECIFIC EXAMPLES

### General Experimental Conditions

### HPLC Method 1:

HPLCs were acquired on a Waters Alliance 2695 LC system. Column: Kromasil C18, 5 μm, 4.6×250 mm. Flow rate: 1 mL/min. Detector: UV, 254 nm. Mobile phase A: (33:9.5:57.5; v/v/v) 1-octanesulfonic acid sodium salt solution/glacial acetic acid/methanol. The 1-octanesulfonic acid sodium salt solution was prepared dissolving 1.6 g of 1-octanesulfonic acid sodium salt in 330 ml of water and adding 95 ml of glacial acetic acid and 575 ml of methanol. Mobile phase B: methanol. Gradient: 100% A (0-20 min)-85% A (40-65 min)-100% A (70-80 min). Temperature: 30° C. Sample: 10 mg/mL in mobile phase A. Injection volume: 10 μL.

Approximate Retention Time for chlorhexidine: 25 minutes.

Approximate Retention Time for p-chloroaniline: 5 minutes.

Limit of detection (LOD): 3 ppm of p-chloroaniline.

### HPLC Method 2:

HPLCs were acquired on a Waters Alliance 2695 LC system. Column: Nucleosil C18, 10 μm, 4.0 cm×200 mm. Flow rate: 1 mL/min. Detector: UV, 254 nm. Mobile phase: (42:9.5:108; v/v/v) 1-octanesulfonic acid sodium salt solution in water/glacial acetic acid/methanol. The 1-octanesulfonic acid sodium salt solution was prepared dissolving 3.0 g of 1-octanesulfonic acid sodium salt in a mixture of 95 ml of glacial acetic acid, 420 ml of water and 1080 ml of methanol. Temperature: room temperature (20-25° C.). Sample: 1 mg/mL in mobile phase. Injection volume: 20 μm.

Approximate Retention Time for chlorhexidine: 7 minutes.

### Examples 1-5

### Comparative study of purifications of 1,1′-hexamethylenebis[5-(4-chlorophenyl)biguanidine], i.e. chlorhexidine base

General Procedure. 2.0 g (3.96 mmol) of chlorhexidine [residual content of p-chloroaniline: 776 ppm (HPLC method 1)] was suspended in 5.76 mL (Examples 1, 4, and 5) or in 12.66 mL (Examples 2 and 5) of the solvent. The resulting suspension was stirred 1 h at room temperature. The white solid was filtered and washed with the solvent and wet Chlorhexidine base was obtained. The solid was dried 5 h at 60° C. and dry chlorhexidine base was obtained. The content of p-chloronailine was determined (HPLC method 1). The results are summarized in Table 2 below.

TABLE 2

| | | Residual con- | Residual | Percent- |
|---|---|---|---|---|
| | | tent of (III) | content of | age of |
| | | in starting | (III) in puri- | reduc- |
| | | chlor- | fied chlor- | tion of |
| Example | Solvent | hexidine | hexidine | (III) |
| Comparative | H₂O | 776 ppm | 728 ppm | 6% |
| Example 1 | | | | |
| Comparative | Isopropanol/ | 776 ppm | 371 ppm | 52% |
| Example 2 | H₂O 20.2:79.8 | | | |
| Comparative | Methanol/ | 776 ppm | 553 ppm | 29% |
| Example 3 | H₂O 25:75 | | | |
| 4 | Isopropanol | 776 ppm | 304 ppm | 61% |
| 5 | Methanol | 776 ppm | 243 ppm | 69% |

### Examples 6-8

### Comparative study of purifications of 1,1′-hexamethylenebis[5-(4-chlorophenyl)biguanidine], i.e. chlorhexidine base

General Procedure: 2.0 g (3.96 mmol) of chlorhexidine [residual content of p-chloroaniline: 776 ppm (HPLC method 1)] was suspended in 5.76 mL of the solvent. The resulting suspension was heated to reflux temperature and was stirred 1 h. Then, the suspension was cooled down to 5° C. and was stirred 3 h. The white solid was filtered and washed with the solvent and wet chlorhexidine base was obtained. The solid was dried 5 h at 60° C. and dry chlorhexidine base was obtained. The content of p-chloroaniline was determined (HPLC method 1). The results are summarized in Table 3 below.

TABLE 3

| | | Residual | Residual | Percent- |
|---|---|---|---|---|
| | | content of (III) | content of (III) | age of |
| | | in starting | in purified | reduction |
| Example | Solvent | chlorhexidine | chlorhexidine | of (III) |
| Comparative | H₂O | 776 ppm | 899 ppm | −16% |
| Example 6 | | | | |
| 7 | Isopropanol | 776 ppm | 210 ppm | 73% |
| 8 | Methanol | 776 ppm | 86 ppm | 89% |

### Example 9

### Purification of 1,1′-hexamethylenebis[5-(4-chlorophenyl) biguanidine], i.e. chlorhexidine base

32.1 kg of chlorhexidine [residual content of (III): 776 ppm (HPLC method 1)] was suspended in a mixture of 72.5 kg (91.5 L) of methanol and 11.76 kg (14.9 L) of acetone. The resulting suspension was heated to reflux temperature and was stirred 1 h. Then, the suspension was cooled down to 15° C. and was stirred 3 h. The white solid was filtered and washed with methanol (3×10 kg) and 31.8 kg of wet chlorhexidine base was obtained. The solid was dried 5 h at 60° C. and 5 h at 80° C., and 25.4 kg of dry chlorhexidine base was obtained (Yield: 79%).

Analytical Data:

Purity (HPLC method 2, % Area): 99.06%, Total impurities: 0.65%, Max. Ind. Impurity: 0.28%. p-Chloroaniline (III) content (HPLC method 1): 132 ppm. Percentage of reduction of (III): 83%.

### Example 10

### Purification of 1,1′-hexamethylenebis[5-(4-chlorophenyl)biguanidine], i.e. chlorhexidine base.

37.6 g of wet chlorhexidine obtained in example 9 (loss on drying=20.14%, 30.0 g dry equivalent, 59.3 mmol) was suspended in 118.9 g (150 mL) of methanol. The resulting suspension was heated to reflux temperature and was stirred 1 h. Then, the suspension was cooled down to 20-25° C. and was stirred 2 h. The white solid was filtered and washed with methanol (3×15 mL), and 34.12 g of wet chlorhexidine base was obtained.

The wet chlorhexidine base was again suspended in 118.9 g (150 mL) of methanol. The resulting suspension was heated to reflux temperature and was stirred 1 h. Then, the suspension was cooled down to 20-25° C. and was stirred 2 h at this temperature. The white solid was filtered and washed with methanol (3×15 mL), and 29.92 g of wet chlorhexidine base was obtained. The solid was dried 5 h at 50° C. and 5 h at 80° C. and 25.22 g of pure chlorhexidine base was obtained (Yield 90%).

Analytical Data:

Purity (HPLC method 2, % Area): 99.86%, Total impurities: 0.03%, Max. Ind. Impurity: 0.03%. p-Chloroaniline (III) content (HPLC method 1): 30 ppm. Total percentage of reduction of (III): 77%. M.p.=134.6-135.4° C.

### Example 11

### Studies of accelerated degradation of chlorhexidine to p-chloroaniline

Two 100 mg aliquots of a sample of chlorhexidine free of potential genotoxicity (Replicates A and B), having a content of p-chloroaniline below 40-50 ppm, a content of each of compounds of Markush formula (IV) (i.e. impurities B-G) below 0.15%, and a total impurities content below 1.5%, as measured by HPLC method 1, were dissolved in 1 mL of a mixture of methanol and acetic acid 8:1 (v/v), and each solution was stored in 5 different closed glass vials at 40° C. These vials were opened after 1, 2, 4, 14, and 25 days of storage, respectively, and the content of p-chloroaniline was measured (HPLC method 1). The obtained results are shown in Table 1 below. A linear regression line was calculated from the data obtained in Table 1 (y=2170x-356, R²=0.991, where “y” represents the measured content of p-chloro aniline in ppm units and “x” represents the degradation time in day units; See FIG. 1). The linear regression line shows the degradation rate towards p-chloroaniline of chlorhexidine free of potential genotoxicity, having less than 0.15% of impurities B-G, and having a total impurities content less than 1.5%, stored in a methanol:acetic acid 8:1 (v/v) solution at 40° C.

TABLE 1

| | Days | 0 | 1 | 2 | 4 | 14 | 25 |
|---|---|---|---|---|---|---|---|
| p-chloro- | Replicate | n.d. | 1,556 | 3,357 | 6,284 | 33,753 | 52,561 |
| aniline (ppm, | A ⁽¹⁾ | | | | | | |
| HPLC | Replicate | n.d. | 1,925 | 3,609 | 7,819 | 33,575 | 51,656 |
| method 1) | B ⁽¹⁾ | | | | | | |
| | Average | — | 1,741 | 3,483 | 7,052 | 33,664 | 52,109 |
| | Standard | — | 261 | 178 | 1,085 | 126 | 640 |
| | deviation | | | | | | |

⁽¹⁾ Prepared from a sample of chlorhexidine free of potential genotoxicity containing
0.142% of compound B, 0.082% of compound C, 0.037% of compound E, 0.140% of
compound G, and 0.54% of total impurities (HPLC, method 1); n.d.: not detected (i.e.
lower than 3 ppm, HPLC method 1).

Three 100 mg aliquots of a sample of chlorhexidine (Replicates C, D and E), having a content of p-chloroaniline of less than 40-50 ppm, a content of each of compounds of Markush formula (IV) (i.e. impurities B-G) higher than 0.15%, and a total impurities content below 2.5-3.0%, as measured by HPLC method 1, were dissolved in 1 mL of a mixture of methanol and acetic acid 8:1 (v/v), and each solution was stored in 2 different closed glass vials at 40° C. These vials were opened after 12 and 19 days of storage, respectively, and the content of p-chloroaniline was measured (HPLC method 1). The obtained results are shown in Table 2 below.

TABLE 2

| | Days | 0 | 12 | 19 |
|---|---|---|---|---|
| p-chloroaniline | Replicate C ⁽¹⁾ | n.d. | 33657 | 45243 |
| (ppm, HPLC | Replicate D ⁽¹⁾ | n.d. | 33153 | 46753 |
| method 1) | Replicate E ⁽¹⁾ | n.d. | 33568 | 47577 |
| | Average | — | 33459 | 46524 |
| | Standard deviation | — | 269 | 1184 |

⁽¹⁾Prepared from a sample of chlorhexidine free of potential genotoxicity containing 0.49% of compound B, 0.54% of compound C, 0.32% of compound E, 0.22% of compound G, and 2.64% of total impurities (HPLC, method 1); n.d.: not detected (i.e. lower than 3 ppm, HPLC method 1).

The two values showing the content of p-chloroaniline after 12 and 19 days of storage in a methanol:acetic acid 8:1 (v/v) solution at 40° C. of a sample of chlorhexidine being initially free of potential genotoxicity, but having a content of compounds of Markush formula (IV) (i.e. impurities B-G) higher than 0.15%, and a total impurities content between 1.5-3.0%, as measured by HPLC method 1, are visualized in FIG. 1, and demonstrate that said chlorhexidine show a higher rate of degradation into p-chloroaniline as compared with the chlorhexidine free of potential genotoxicity having a content of each of compounds of Markush formula (IV) (i.e. impurities B-G) below 0.15%, and a total impurities content below 1.5%, as measured by HPLC method 1, when stored in a methanol:acetic acid 8:1 (v/v) solution at 40° C.

### Example 12

### Stability Study of Aqueous Solutions of Chlorhexidine Digluconate

Two samples corresponding to a 20% (v/w) aqueous solution of chlorhexidine digluconate which was prepared from the chlorhexidine of Example 10, were stored in a closed vial, under different temperature conditions (i.e. 15° C., 25° C., and 40° C.) for 3 months in a closed chamber protected from light. The potentially free genotoxicity stability of the samples (i.e. the presence of p-chloroaniline) was measured by the colorimetric method described for measuring p-chloroaniline in chlorhexidine digluconate solution in European Pharmacopoeia 6.0 (page 1501). The obtained results are shown in Table 3 below.

TABLE 3

| Temperature of | p-chloroaniline (ppm, Colorimetry) | | |
|---|---|---|---|
| storage (° C.) | Initial | 3 months | Increasing (%) |
| 15° C. | 3 ppm | 16 ppm | 433% |
| 25° C. | 3 ppm | 49 ppm | 1533% |
| 40° C. | 3 ppm | 130 ppm | 4233% |

1. Chlorhexidine, (compound of formula I), 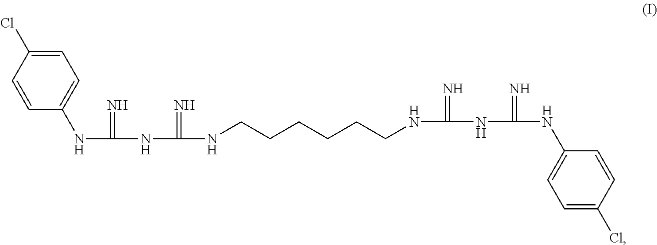 [SMILES: N=C(NCCCCCCNC(=N)NC(=N)Nc1ccc(Cl)cc1)NC(=N)Nc1ccc(Cl)cc1]
or a pharmaceutically acceptable salt thereof, which is free of potential genotoxicity, and wherein the said chlorhexidine, or a pharmaceutically acceptable salt thereof, has a concentration of equal to or less than about 40-50 ppm with respect to chlorhexidine, by HPLC, of p-chloroaniline (compound of formula III),

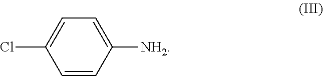

2. The chlorhexidine, or a pharmaceutically acceptable salt thereof, of claim 1, which has a content of equal to or less than 0.15% of percentage area by HPLC of each of the compounds of formula IV, 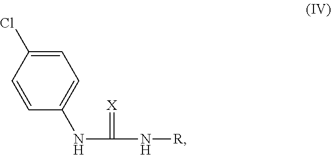
wherein X is NH or O, and R is H, a carboxamidino group, or a carboxamido group.

3. The chlorhexidine, or a pharmaceutically acceptable salt thereof, of claim 2, wherein the compound of formula (IV) is selected from the group consisting of compounds B-G,

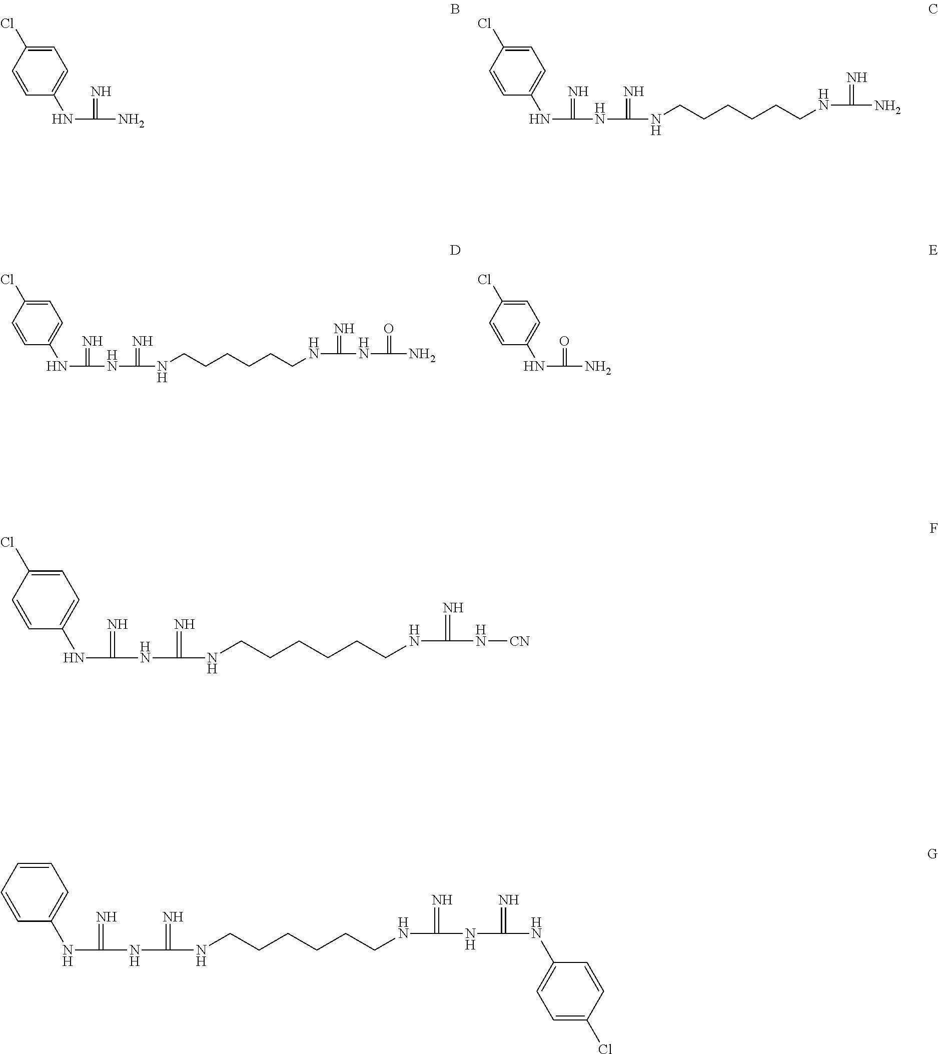 [SMILES: N=C(N)Nc1ccc(Cl)cc1 ; N=C(N)NCCCCCCNC(=N)NC(=N)Nc1ccc(Cl)cc1 ; N=C(NCCCCCCNC(=N)NC(N)=O)NC(=N)Nc1ccc(Cl)cc1 ; NC(=O)Nc1ccc(Cl)cc1 ; N#CNC(=N)NCCCCCCNC(=N)NC(=N)Nc1ccc(Cl)cc1 ; N=C(NCCCCCCNC(=N)NC(=N)Nc1ccc(Cl)cc1)NC(=N)Nc1ccccc1]

4. The chlorhexidine, or a pharmaceutically acceptable salt thereof, of claim 1, which is the mono- or diacetate salt, the mono- or dihydrochloride salt, or the mono- or digluconate salt of chlorhexidine.

5. A pharmaceutical composition comprising the chlorhexidine or a pharmaceutically acceptable salt thereof as defined in claim 1, together with at least one pharmaceutically carrier and/or with at least one additional active pharmaceutical ingredient.

6. The pharmaceutical composition of claim 5, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.

7.-29. (canceled)

30. The chlorhexidine, or a pharmaceutically acceptable salt thereof, of claim 2, which is the mono- or diacetate salt, the mono- or dihydrochloride salt, or the mono- or digluconate salt of chlorhexidine.

31. The chlorhexidine, or a pharmaceutically acceptable salt thereof, of claim 3, which is the mono- or diacetate salt, the mono- or dihydrochloride salt, or the mono- or digluconate salt of chlorhexidine.

32. A pharmaceutical composition comprising the chlorhexidine or a pharmaceutically acceptable salt thereof as defined in claim 2, together with at least one pharmaceutically carrier and/or with at least one additional active pharmaceutical ingredient.

33. The pharmaceutical composition of claim 32, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.

34. A pharmaceutical composition comprising the chlorhexidine or a pharmaceutically acceptable salt thereof as defined in claim 3, together with at least one pharmaceutically carrier and/or with at least one additional active pharmaceutical ingredient.

35. The pharmaceutical composition of claim 34, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.

36. A pharmaceutical composition comprising the chlorhexidine or a pharmaceutically acceptable salt thereof as defined in claim 4, together with at least one pharmaceutically carrier and/or with at least one additional active pharmaceutical ingredient.

37. The pharmaceutical composition of claim 36, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.

38. A pharmaceutical composition comprising the chlorhexidine or a pharmaceutically acceptable salt thereof as defined in claim 30, together with at least one pharmaceutically carrier and/or with at least one additional active pharmaceutical ingredient.

39. The pharmaceutical composition of claim 38, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.

40. A pharmaceutical composition comprising the chlorhexidine or a pharmaceutically acceptable salt thereof as defined in claim 31, together with at least one pharmaceutically carrier and/or with at least one additional active pharmaceutical ingredient.

41. The pharmaceutical composition of claim 40, wherein the at least one pharmaceutically acceptable carrier is benzalkonium chloride, menthol, ethanol, water, or mixtures thereof, and wherein the at least one additional active pharmaceutical ingredient is benzocaine, tirotricine, lidocaine, enoxolone, or mixtures thereof.
